(19) United States
(12) Patent Application Publication
(10) Pub. No.: US 2025/0340582 A1
(43) Pub. Date: Nov. 6, 2025

(54) CATALYST FOR SELECTIVELY REDUCING SPINOSYN J AND PROCESS USING SAME

(71) Applicant: CANAN TECHNIQUE MATERIAL (HANGZHOU) INC, Hangzhou, Jhejiang (CN)

(72) Inventors: Dingjun WANG, Hangzhou, Jhejiang (CN); Xue SONG, Hangzhou, Jhejiang (CN); Honghua LI, Hangzhou, Jhejiang (CN); Jiayou ZHANG, Hangzhou, Jhejiang (CN)

(21) Appl. No.: 18/873,472

(22) PCT Filed: Dec. 31, 2022
(86) PCT No.: PCT/CN2022/144389

§ 371 Date: Dec. 10, 2024

(30) Foreign Application Priority Data
Jun. 10, 2022 (CN) 202210658057.5

(51) Int. Cl. C07H 17/08; B01J 21/04; B01J 21/06; B01J 21/08; B01J 21/18; B01J 23/42; B01J 23/44; B01J 23/46; B01J 27/232; B01J 35/61; C07H 1/00

(52) U.S. Cl. CPC C07H 17/08; B01J 21/04; B01J 21/063; B01J 21/08; B01J 21/18; B01J 23/42; B01J 23/44; B01J 23/462; B01J 23/464; B01J 23/468; B01J 27/232; B01J 35/613; B01J 35/615; B01J 35/617; B01J 35/618; C07H 1/00

(57) ABSTRACT

The present application relates to the technical field of spinosyn preparation, in particular to a catalyst for selectively reducing spinosyn J. The weight of the active component in the catalyst accounts for 1%-10% of the weight of the catalyst, and the active component is one selected from Pd, Pt, Rh, Ru and Ir; the active component comprises at least two metals, a first metal is selected from the group consisting of Pd, Pt or Rh, and the remaining metal is any one or more than one selected from the group consisting of Pd, Pt, Ru and Ir. The catalyst can reduce a 5,6-position double bond on a spinosyn J four-membered ring lactone in a spinosyn J/L mixture by reacting with hydrogen under the conditions of 0.05 MPa-0.5 MPa and 10° C.-80° C. for 5 h-10 h, without accompanying reduction of a 13,14-position double bond on the spinosyn J four-membered ring lactone and spinosyn L. The catalyst conversion rate, selectivity and yield of the present application are higher, and the cost is lower.

### TECHNICAL FIELD

The present disclosure relates to the technical field of spinosyn preparation, in particular to a catalyst for selectively reducing spinosyn J and a process using the same. The selective reduction is specifically to catalyze and reduce a 5,6-position double bond on a spinosyn J four-membered ring lactone in a spinosyn J/L mixture, without accompanying reduction of a 13,14-position conjugated double bond on the spinosyn J four-membered ring lactone and spinosyn L.

### BACKGROUND ART

The spinosyns are a kind of intracellular secondary metabolite produced by the Gram-positive, aerobic soil actinomycete Saccharopolyspora spinosa through aerobic fermentation. The spinosyns are macrolides in structure and consist of two deoxysugars (trioxymethyl rhamnose and flexosamine) attached to a 21C four-membered ring lactone. The main components in the fermentation products of Saccharopolyspora spinosa are spinosyn A and spinosyn D, which are collectively called spinosad.

After strain modification, the fermentation product of Saccharopolyspora spinosa is changed into a mixture mainly containing spinosyn J/L. The difference between spinosyn J and spinosyn L lies in whether the substituent at the 6th carbon position of the four-membered ring lactone is hydrogen or methyl. After a biological fermentation to obtain primary products, selective hydrogenation reduction of a mixture of spinosyn J/L is required. Specifically, the 5,6-position double bond on a spinosyn J four-membered ring lactone is selectively reduced and the 13, 14-position conjugated double bond is not reduced to thereby obtain 5,6-dihydro-spinosyn J, and spinosyn L cannot be reduced.

At present, there are few reports on the catalyst for selective reduction of spinosyn J/L, and the representative one is a Chinese patent application publication numbered CN101535330A, with a title of “Selective reduction of spinosyn factors ET-J and ET-L to spinetoram”, which discloses hydrogenation of a mixture of 3′-O-ethyl-spinosyn J/L with hydrogen in the presence of heterogeneous catalyst 5% Rh/Al2O3, 5% Pd/C capable of selectively reducing the 5,6-position double bond of 3′-O-ethyl-spinosyn J in a water-miscible organic solvent until all 3′-O-ethyl-spinosyn J is converted to 3′-O-ethyl-5,6-dihydro-spinosyn J. For the catalyst Rh/Al2O3 proposed in this patent application, due to the high consumption of the noble metal rhodium, the production cost of the product is too high, which is not conducive to large-scale application. However, the selectivity of 5% Pd/C catalyst is not high and the reaction time is too long, resulting in hydrogenation of 3′-O-ethyl-spinosyn L and lower yield, which is also undesirable. In addition, according to the patent application, spinosyn is first ethylated and then goes through hydrogenation reduction under the action of a heterogeneous catalyst, resulting in too long reaction time, which is not conducive to reducing production costs, and unforeseen side reactions may occur after a long-term reaction.

SUMMARY

In view of the technical problems existing in the prior art, the purpose of this patent application is to provide a catalyst with higher activity and better selectivity. In addition, the patent application also provides a process method for preparing 3′-O-ethyl-5,6-dihydro-spinosyn J and 3′-O-ethyl-spinosyn L. Based on the catalyst proposed in the patent application, the reaction time is greatly reduced, which is a more efficient, greener and more economical process.

A catalyst for selectively reducing spinosyn J is used in a water-miscible organic solvent. The catalyst is used for selectively catalyzing and reducing a 5,6-position double bond on a spinosyn J four-membered ring lactone in a spinosyn J/L mixture, without accompanying reduction of a 13,14-position conjugated double bond on the spinosyn J four-membered ring lactone and spinosyn L. The catalyst comprises an active component and a carrier, the weight of the active component in the catalyst accounts for 1%- 10% of the weight of the catalyst, and the active component is selected from a group consisting of Pd, Pt, Rh, Ru and Ir. In particular, the active component comprises two metals, one metal being selected from a group consisting of Pd, Pt or Rh and the other metal being selected from a group consisting of Pd, Pt, Ru or Ir; or the active component comprises three or more metals, which are respectively a first active metal selected from any one of Pd, Pt and Rh, a second active metal selected from any one of Pd, Pt, Ru and Ir, and a secondary active metal selected from one or more of Pd, Pt, Ru and Ir. The weight ratio of the first active metal, the second active metal to the secondary active metal is (6-12):(6-13):(1-2).

Further, the active component accounts for 3%-6% by weight of the catalyst, and the specific surface area of the catalyst carrier is 50 m²/g-2,000 m²/g.

Further, the carrier is selected from a group consisting of activated carbon, graphite, carbon black, alumina, CaCO₃, ZrO₂, TiO₂, SiO₂ and diatomaceous earth.

Further, the carrier of the catalyst is coal carbon, lignocellulose carbon, coconut shell carbon or γ-alumina.

Further, in Step \textcircled{1}, reactant mixing: adding a spinosyn J/L mixture, an organic solvent and water to a reactor for mixing, then adding the catalyst according to claim 1, stirring and dissolving them in such a manner that the weight ratio of spinosyn J/L mixture, organic solvent to water is (10-50):(50-100):(1-5), and the weight ratio of the dry catalyst to the spinosyn J/L mixture is (1-5):100; Step \textcircled{2}, gas replacement: introducing nitrogen to the reactor to replace air, and then replacing nitrogen with hydrogen; Step \textcircled{3}, hydrogenation: completing the reduction of the 5,6-position double bond on a spinosyn J four-membered ring lactone by reacting in a hydrogen atmosphere under the conditions of 0.05 MPa-0.5 MPa and 10° C.-80° C. for 5 h-10 h.

Further, the organic solvent is selected from a group consisting of toluene, ethyl acetate, methanol, ethanol, isopropanol, tert-butyl methyl ether, tetrahydrofuran, glycol ethers, acetonitrile and acetone; and the weight ratio of spinosyn J/L mixture, organic solvent to water is (20-30):(70-80):(1-5).

Further, the gas replacement in Step \textcircled{2} is specifically as follows: firstly, replacing air in the kettle with high-purity nitrogen at least once, and then replacing the nitrogen in the kettle with high-purity hydrogen at least once.

Furthermore, during the hydrogenation in Step \textcircled{3}, the reaction is carried out under the conditions of 0.1 MPa-0.3 MPa and 25° C.-50° C.

Further, the hydrogenation materials in Step \textcircled{3}, the hydrogenation, tetrabutylammonium bromide, potassium hydroxide and water are added to a reactor, and the mixture is fully stirred to dissolve and then sealed in the reactor; nitrogen is introduced to the reactor to replace air in the reactor at least once; after adding ethyl bromide, the mixture inside the reactor is heated up to 40° C., the pressure rises to 0.3 Mpa by supplementing nitrogen, and the stirring speed is adjusted to 300 r/min; under these conditions, the mixture reacts for 5 h, is cooled down to room temperature, transferred to an enamel kettle, added with diethyl ether for crystallization, filtered, and dried at 60° C. to obtain spinetoram, a final product.

Further, the spinosyn J/L mixture in Step \textcircled{1} reactant mixing is a 3′-O-ethyl-spinosyn J/L mixture.

The above technical solution has the following advantages or beneficial effects: the catalyst conversion rate, selectivity and yield of the present application are higher, and the cost is lower.

### DETAILED DESCRIPTION OF THE PREFERRED EMBODIMENTS

Embodiments are described in detail below, which are exemplary and intended to be used for explaining the inventive concept.

The selective catalytic reduction of a 5,6-position double bond on a spinosyn J four-membered ring lactone without accompanying reduction of a 13,14-position conjugated double bond to obtain 5,6-dihydro-spinosyn J, and spinosyn L cannot be reduced during hydrogenation of spinosyn J. The 3′-position of rhamnose is then ethylated. The chemical theory of the present patent application is as follows:

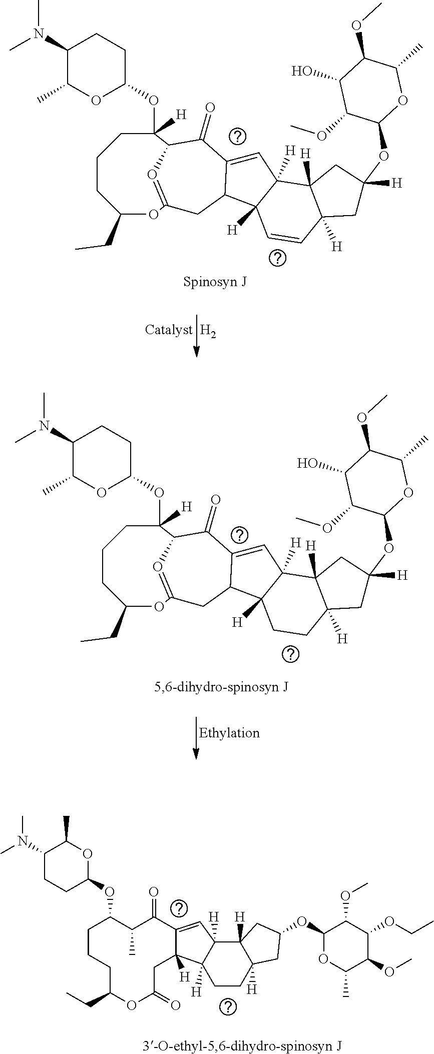

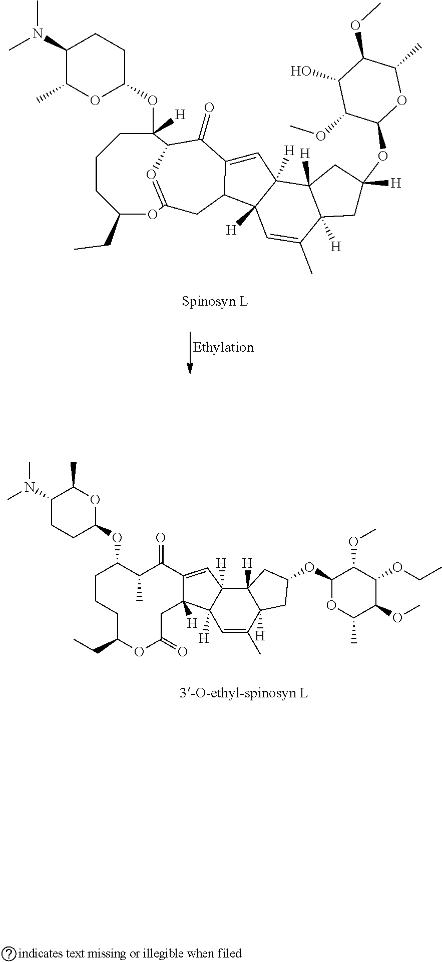

The catalyst for selective catalytic reduction of spinosyn J comprises an active component and a carrier, and the mass ratio of the active component to the carrier is (20-0.1):(80-99.9). Preferably, the active component accounts for 1%- 10% by weight of the catalyst, more preferably 3%- 6%. Upon a loading amount greater than 20%, the hydrogenation effect is still effective, but it will cause a decrease in activity per unit metal content and increase the cost. The active component of the catalyst accounts for 1%-10% by weight of the catalyst, and the active component is selected from a group consisting of Pd, Pt, Rh, Ru and Ir. In particular, the active component is two metals, one metal being selected from a group consisting of Pd, Pt or Rh and the other metal being selected from a group consisting of Pd, Pt, Ru or Ir; the active component comprises three or more metals, which are respectively a first active metal selected from any one of Pd, Pt and Rh, a second active metal selected from any one of Pd, Pt, Ru and Ir, and a secondary active metal selected from one or more of Pd, Pt, Ru and Ir. The weight ratio of the first active metal, the second active metal to the secondary active metal is (6-12):(6-13):(1-2).

The catalyst carrier is one of activated carbon, graphite, carbon black, alumina, CaCO₃, ZrO₂, TiO₂, SiO₂, diatomaceous earth and other porous support materials that can be used for supporting. Among them, activated carbon includes but is not limited to coal carbon, lignocellulose carbon and coconut shell carbon; alumina can be in various crystal forms, with γ-alumina being the best. The catalyst carrier has a specific surface area of 50 m²/g-2,000 m²/g.

The raw material of this application is a spinosyn J/L mixture, and the solvent can be any organic solvent that effectively dissolves spinosyn J/L, such as toluene, ethyl acetate, alcohols (methanol, ethanol, isopropanol), ethers (tert-butyl methyl ether, tetrahydrofuran), glycol ethers, acetonitrile and acetone.

### Embodiment 1 Hydrogenation of Solid Spinosyn Factor Spinosyn J/L Mixture with 5% Pd/CaCO₃

Add 220 g of spinosyn J/L mixture and 800 g of isopropanol to a 2 L reactor in turn, add 40 g of water, then add 4.4 g (2% by weight of spinosyn J/L mixture) of 5% Pd/CaCO₃ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H2 and release it. Repeat this process for 5 times. Finally, pressurize the reactor with H2 to 0.15 MPa and what inside the reactor reacts for 7 h under stirring. After filtration and spin-drying, use liquid chromatography to test what inside the reactor. The hydrogenation of spinosyn J is completed with a conversion rate of 99%, a selectivity of 99%, and a product yield of higher than 90.1%. No peak of 5,6-dihydro-spinosyn L is observed on the liquid chromatogram, indicating that the hydrogenation rate of spinosyn L is 0.

Specific ethylation method: Pour the above hydrogenated materials into the reactor again, add 50 g of tetrabutylammonium bromide and 100 g of potassium hydroxide, add 700 g of water, stir them to dissolve, and seal the reactor cover. Pressurize the reactor to 0.5 Mpa with nitrogen and then release the air to replace it. Repeat this process for 5 times. After adding 30 g of bromoethane, heat the mixture up to 40° C., supplement the pressure to 0.3 Mpa with nitrogen, and adjust the rotation speed to 300 r/min; the mixture inside the reactor reacts for 5 h under this condition, cool the mixture down to room temperature, transfer it into an enamel kettle, add diethyl ether for crystallization, filter the mixture to obtain an ethylated product, and dry it at 60° C. to obtain the final product.

### Embodiment 2 Hydrogenation of Solid Spinosyn Factor Spinosyn J/L Mixture with 5% Pt/C

Add 200 g of spinosyn J/L mixture and 800 g of acetone to a 2 L reactor in turn, add 16 g of water, then add 6 g (3% by weight of spinosyn J/L mixture) of 5% Pt/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.18 MPa and what inside the reactor reacts for 7 h under stirring. After filtration and spin-drying, use liquid chromatography to test what inside the reactor. The hydrogenation of spinosyn J is completed with a conversion rate of 99%, a selectivity of 99%, and a product yield of higher than 91.5%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 3 Hydrogenation of Solid Spinosyn Factor Spinosyn J/L Mixture with 2% Rh3% Ru/TiO₂

Add 250 g of spinosyn J/L mixture and 750 g of isopropanol to a 2 L reactor in turn, add 15 g of water, then add 7.5 g (3% by weight of spinosyn J/L mixture) of 2% Rh3% Ru/TiO₂ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.1 MPa and what inside the reactor reacts for 5 h under stirring. After filtration and spin-drying, use liquid chromatography to test what inside the reactor. The hydrogenation of spinosyn J is completed with a conversion rate of 99%, a selectivity of 99%, and a product yield of higher than 90.9%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 4 Hydrogenation of Solid Spinosyn Factor Spinosyn J/L Mixture with 2% Pd3% Pt/C

Add 300 g of spinosyn J/L mixture and 700 g of ethanol to a 2 L reactor in turn, add 21 g of water, then add 6 g (2% by weight of spinosyn J/L mixture) of 2% Pd3% Pt/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 9 h under stirring. After filtration and spin-drying, use liquid chromatography to test what inside the reactor. The hydrogenation of spinosyn J is completed with a conversion rate of 99%, a selectivity of 99%, and a product yield of higher than 92.8%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 5 Hydrogenation of Solid Spinosyn Factor Spinosyn J/L Mixture with 2% Rh3% Pd/Al₂O₃

Add 250 g of spinosyn J/L mixture and 750 g of isopropanol to a 2 L reactor in turn, add 20 g of water, then add 5 g (2% by weight of spinosyn J/L mixture) of 2% Rh3% Pd/Al₂O₃ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 8 h under stirring. After filtration and spin-drying, use liquid chromatography to test what inside the reactor. The hydrogenation of spinosyn J is completed with a conversion rate of 99%, a selectivity of 99%, and a product yield of higher than 93.1%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 6 Hydrogenation of Solid Spinosyn Factor Spinosyn J/L Mixture with 2% Pd3% Ru/C

Add 250 g of spinosyn J/L mixture and 750 g of isopropanol to a 2 L reactor in turn, add 20 g of water, then add 7.5 g (3% by weight of spinosyn J/L mixture) of 2% Pd3% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.2 MPa and what inside the reactor reacts for 10 h under stirring. After filtration and spin-drying, use liquid chromatography to test what inside the reactor. The hydrogenation of spinosyn J is completed with a conversion rate of 99%, a selectivity of 99%, and a product yield of higher than 90.4%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 7 Hydrogenation of Solid Spinosyn Factor Spinosyn J/L Mixture with 3% Rh3% Pt/CaCO₃

Add 250 g of spinosyn J/L mixture and 750 g of tetrahydrofuran to a 2 L reactor in turn, add 20 g of water, then add 5 g (2% by weight of spinosyn J/L mixture) of 3% Rh3% Pt/CaCO₃ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.25 MPa and what inside the reactor reacts for 7 h under stirring. After filtration and spin-drying, use liquid chromatography to test what inside the reactor. The hydrogenation of spinosyn J is completed with a conversion rate of 99%, a selectivity of 99%, and a product yield of higher than 91.6%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 8 Hydrogenation of Solid Spinosyn Factor Spinosyn J/L Mixture with 2% Rh3% Ru/Al₂O₃

Add 300 g of spinosyn J/L mixture and 750 g of acetone to a 2 L reactor in turn, add 20 g of water, then add 9 g (3% by weight of spinosyn J/L mixture) of 2% Rh3% Ru/Al₂O₃ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 8 h under stirring. After filtration and spin-drying, use liquid chromatography to test what inside the reactor. The hydrogenation of spinosyn J is completed with a conversion rate of 99%, a selectivity of 99%, and a product yield of higher than 95.1%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 9 Hydrogenation of Solid Spinosyn Factor Spinosyn J/L Mixture with 1% Rh2% Ru/C

Add 250 g of spinosyn J/L mixture and 750 g of acetone to a 2 L reactor in turn, add 20 g of water, then add 5 g (2% by weight of spinosyn J/L mixture) of 1% Rh2% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.1 MPa and what inside the reactor reacts for 9 h under stirring. After filtration and spin-drying, use liquid chromatography to test what inside the reactor. The hydrogenation of spinosyn J is completed with a conversion rate of 99%, a selectivity of 99%, and a product yield of higher than 90.6%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 10 Hydrogenation of solid spinosyn factor spinosyn J/L mixture with 1% Pd4% Pt/C

Add 200 g of spinosyn J/L mixture and 600 g of toluene to a 2 L reactor in turn, add 20 g of water, then add 5 g (2.5% by weight of spinosyn J/L mixture) of 1% Pd4% Pt/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 7 h under stirring. After filtration and spin-drying, use liquid chromatography to test what inside the reactor. The hydrogenation of spinosyn J is completed with a conversion rate of 99%, a selectivity of 99%, and a product yield of higher than 92.2%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 11 Hydrogenation of Solid Spinosyn Factor Spinosyn J/L Mixture with 3% Rh3% Ru/C

Add 250 g of spinosyn J/L mixture and 750 g of isopropanol to a 2 L reactor in turn, add 20 g of water, then add 5 g (2% by weight of spinosyn J/L mixture) of 3% Rh3% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 6 h under stirring. After filtration and spin-drying, use liquid chromatography to test what inside the reactor. The hydrogenation of spinosyn J is completed with a conversion rate of 99%, a selectivity of 99%, and a product yield of higher than 95.6%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 12 Hydrogenation of Solid Spinosyn Factor Spinosyn J/L Mixture with 2% Pt2% Ru/C

Add 250 g of spinosyn J/L mixture and 700 g of acetone to a 2 L reactor in turn, add 20 g of water, then add 6.25 g (2.5% by weight of spinosyn J/L mixture) of 2% Pt2% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 8 h under stirring. After filtration and spin-drying, use liquid chromatography to test what inside the reactor. The hydrogenation of spinosyn J is completed with a conversion rate of 99%, a selectivity of 99%, and a product yield of higher than 93.4%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 13 Hydrogenation of Solid Spinosyn Factor Spinosyn J/L Mixture with 1% Pd3% Ir/C

Add 200 g of spinosyn J/L mixture and 800 g of isopropanol to a 2 L reactor in turn, add 20 g of water, then add 5 g (2.5% by weight of spinosyn J/L mixture) of 1% Pd3% Ir/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.2 MPa and what inside the reactor reacts for 9 h under stirring. After filtration and spin-drying, use liquid chromatography to test what inside the reactor. The hydrogenation of spinosyn J is completed with a conversion rate of 99%, a selectivity of 99%, and a product yield of higher than 90.9%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 14 Hydrogenation of Solid Spinosyn Factor Spinosyn J/L Mixture with 2% Rh1% Ru/SiO₂

Add 250 g of spinosyn J/L mixture and 750 g of acetone to a 2 L reactor in turn, add 15 g of water, then add 5 g (2% by weight of spinosyn J/L mixture) of 2% Rh1% Ru/SiO2 (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.1 MPa and what inside the reactor reacts for 10 h under stirring. After filtration and spin-drying, use liquid chromatography to test what inside the reactor. The hydrogenation of spinosyn J is completed with a conversion rate of 99%, a selectivity of 99%, and a product yield of higher than 91.1%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 15 Hydrogenation of Solid Spinosyn Factor Spinosyn J/L Mixture with 2% Pt2% Ru0.5% Ir/TiO₂

Add 250 g of spinosyn J/L mixture and 750 g of acetone to a 2 L reactor in turn, add 20 g of water, then add 7.5 g (3% by weight of spinosyn J/L mixture) of 2% Pt2% Ru0.5% Ir/TiO₂ (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 10 h under stirring. After filtration and spin-drying, use liquid chromatography to test what inside the reactor. The hydrogenation of spinosyn J is completed with a conversion rate of 99%, a selectivity of 99%, and a product yield of higher than 89.4%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 16 Hydrogenation of Solid Spinosyn Factor Spinosyn J/L Mixture with 2% Pt2% Pd0.5% Ru/C

Add 250 g of spinosyn J/L mixture and 750 g of acetone to a 2 L reactor in turn, add 20 g of water, then add 7.5 g (3% by weight of spinosyn J/L mixture) of 2% Pt2% Pd0.5% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.2 MPa and what inside the reactor reacts for 9 h under stirring. After filtration and spin-drying, use liquid chromatography to test what inside the reactor. The hydrogenation of spinosyn J is completed with a conversion rate of 99%, a selectivity of 99%, and a product yield of higher than 87.5%. No peak of 5,6-dihydro-spinosyn L is observed on the liquid chromatogram, indicating that the hydrogenation rate of spinosyn L is 0. The subsequent ethylation process is the same as that in Embodiment 1.

Embodiment 17 Hydrogenation of Solid Spinosyn Factor Spinosyn J/L Mixture with 2% Pt1% Ru0.2% Ir/C

Add 200 g of spinosyn J/L mixture and 800 g of acetone to a 2 L reactor in turn, add 15 g of water, then add 6.0 g (3% by weight of spinosyn J/L mixture) of 2% Pt1% Ru0.2% Ir/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.25 MPa and what inside the reactor reacts for 8 h under stirring. After filtration and spin-drying, use liquid chromatography to test what inside the reactor. The hydrogenation of spinosyn J is completed with a conversion rate of 99%, a selectivity of 99%, and a product yield of higher than 89.5%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 18 Hydrogenation of Solid Spinosyn Factor Spinosyn J/L Mixture with 3% Pd2% Ru0.4% Ir/C

Add 250 g of spinosyn J/L mixture and 700 g of isopropanol to a 2 L reactor in turn, add 18 g of water, then add 5.0 g (2% by weight of spinosyn J/L mixture) of 3% Pd2% Ru0.4% Ir/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.3 MPa and what inside the reactor reacts for 9 h under stirring. After filtration and spin-drying, use liquid chromatography to test what inside the reactor. The hydrogenation of spinosyn J is completed with a conversion rate of 99%, a selectivity of 99%, and a product yield of higher than 90.0%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 19 Hydrogenation of Solid Spinosyn Factor Spinosyn J/L Mixture with 1% Rh2% Ru0.2% Ir/C

Add 250 g of spinosyn J/L mixture and 700 g of isopropanol to a 2 L reactor in turn, add 15 g of water, then add 5.0 g (2% by weight of spinosyn J/L mixture) of 1% Rh2% Ru0.2% Ir/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.15 MPa and what inside the reactor reacts for 10 h under stirring. After filtration and spin-drying, use liquid chromatography to test what inside the reactor. The hydrogenation of spinosyn J is completed with a conversion rate of 99%, a selectivity of 99%, and a product yield of higher than 89.0%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 20 Hydrogenation of Solid Spinosyn Factor Spinosyn J/L Mixture with 1% Rh2% Pt0.2% Ru/C

Add 200 g of spinosyn J/L mixture and 700 g of acetone to a 2 L reactor in turn, add 15 g of water, then add 5.0 g (2.5% by weight of spinosyn J/L mixture) of 1% Rh2% Pt0.2% Ru/C (dry weight) to the solution, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.1 MPa and what inside the reactor reacts for 7 h under stirring. After filtration and spin-drying, use liquid chromatography to test what inside the reactor. The hydrogenation of spinosyn J is completed with a conversion rate of 99%, a selectivity of 99%, and a product yield of higher than 91.0%. No peak of 5,6-dihydro-spinosyn L is observed on the liquid chromatogram, indicating that the hydrogenation rate of spinosyn L is 0. The subsequent ethylation process is the same as that in Embodiment 1.

### Embodiment 21 Hydrogenation of Solid Spinosyn Factor Spinosyn J/L Mixture with 2% Pt2% Ru/C

Ethylation first: Add 200 g of spinosyn J/L mixture, 50 g of tetrabutylammonium bromide and 100 g of potassium hydroxide to a 2 L reactor, add 700 g of water, stir to dissolve, and seal the reactor cover. Pressurize the reactor to 0.5 Mpa with nitrogen and then release the air to replace it. Repeat this process for 5 times. After adding 30 g of bromoethane, heat the mixture up to 40° C., supplement the pressure to 0.3 Mpa with nitrogen, and adjust the rotation speed to 300 r/min; the mixture inside the reactor reacts for 5 h under this condition, cool the mixture down to room temperature, transfer it into an enamel kettle, add diethyl ether for crystallization, filter the mixture to obtain an ethylated product, and dry it at 60° C. to obtain a dried ethylated product 3′-O-ethyl-spinosyn J/L.

Add 200 g of the above 3′-O-ethyl-spinosyn J/L mixture and 800 g of isopropanol to a 2 L reactor in turn, add 40 g of water, then add 4 g (2% by weight of spinosyn J/L mixture) of 2% Pt2% Ru/C to the solution,, and start stirring for dissolution. Tighten the charging port of the reactor, pressurize the kettle to 0.3 MPa with high-purity N₂, and then release the pressure for air replacement in the kettle. Repeat this process for 5 times. Then pressurize the reactor to 0.3 MPa with high purity H₂ and release it. Repeat this process for 5 times. Finally, pressurize the reactor with H₂ to 0.1 MPa and what inside the reactor reacts for 10 h under stirring. After filtration and spin-drying, use liquid chromatography to test what inside the reactor. The hydrogenation of 3′-O-ethyl-spinosyn J is completed with a conversion rate of 99%, a selectivity of 99%, and a product yield of higher than 88.6%. No peak of 3′-O-ethyl-5,6-dihydro-spinosyn L is observed on the liquid chromatogram, indicating that the hydrogenation rate of 3′-O-ethyl-spinosyn L is 0.

Although the embodiments of the present disclosure have been shown and described above, it can be understood that the above embodiments are exemplary for experienced people and cannot be construed as limitations to the present disclosure. Those skilled in the art may make changes, modifications, substitutions and variations to the above embodiments within the scope of the present disclosure without departing from the principle and purpose of the present disclosure. These changes and improvements fall within the claimed scope of this application.

1. A catalyst for selectively reducing spinosyn J, in a water-miscible organic solvent, the catalyst being used for selectively catalyzing and reducing a 5,6-position double bond on a spinosyn J four-membered ring lactone in a spinosyn J/L mixture, without accompanying reduction of a 13, 14-position conjugated double bond on the spinosyn J four-membered ring lactone and spinosyn L, comprising an active component and a carrier, wherein the weight of the active component in the catalyst accounts for 1%- 10% of the weight of the catalyst, and the active component is selected from a group consisting of Pd, Pt, Rh, Ru and Ir; the active component comprises at least two metals, a first metal being selected from a group consisting of Pd, Pt or Rh and the remaining metal being any one or more selected from the group consisting of Pd, Pt, Ru and Ir.

2. The catalyst for selectively reducing spinosyn J according to claim 1, wherein when the active component comprises two metals and the first metal is Rh, a weight ratio of the active component is selected from a group consisting of 1Rh:2Ru, 2Rh:3Ru, 1Rh:1Ru, 2Rh:1Ru, 2Rh:3Pd and 1Rh:1 Pt; when the active component comprises three metals, the weight ratio of the first metal to the remaining two metals is (6-12):(6-13):(1-2).

3. The catalyst for selectively reducing spinosyn J according to claim 1, wherein the active component accounts for 3%- 6% by weight of the catalyst, and the specific surface area of the catalyst carrier is 50 m²/g-2,000 m²/g.

4. The catalyst for selectively reducing spinosyn J according to claim 3, wherein the carrier is selected from a group consisting of activated carbon, graphite, carbon black, alumina, CaCO₃, ZrO₂, TiO₂, SiO₂ and diatomaceous earth.

5. The catalyst for selectively reducing spinosyn J according to claim 4, wherein the carrier of the catalyst is one of coal carbon, lignocellulose carbon, coconut shell carbon or γ-alumina.

6. A process for selectively reducing spinosyn J using the catalyst according to claim 1, wherein the process comprises Step \textcircled{1} reactant mixing: adding a spinosyn J/L mixture, an organic solvent and water to a reactor for mixing, then adding the catalyst according to claim 1, stirring and dissolving them in such a manner that the weight ratio of spinosyn J/L mixture, organic solvent to water is (10-50):(50-100):(1-5), and the weight ratio of the dry catalyst to the spinosyn J/L mixture is (1-5):100; Step \textcircled{2} gas replacement: introducing nitrogen to the reactor to replace air, and then replacing nitrogen with hydrogen; Step 3 hydrogenation: completing the reduction of the 5,6-position double bond on a spinosyn J four-membered ring lactone by reacting in a hydrogen atmosphere under the conditions of 0.05 MPa-0.5 MPa and 10° C.-80° C. for 5 h-10 h.

7. The process for selectively reducing spinosyn J according to claim 6, wherein the organic solvent is selected from a group consisting of toluene, ethyl acetate, methanol, ethanol, isopropanol, tert-butyl methyl ether, tetrahydrofuran, glycol ethers, acetonitrile and acetone; and the weight ratio of spinosyn J/L mixture, organic solvent to water is (20-30):(70-80):(1-5).

8. The process for selectively reducing spinosyn J according to claim 6, wherein the gas replacement in Step \textcircled{2} is specifically as follows: firstly, replacing air in the kettle with high-purity nitrogen at least once, and then replacing the nitrogen in the kettle with high-purity hydrogen at least once.

9. The process for selectively reducing spinosyn J according to claim 6, wherein during the hydrogenation in Step \textcircled{3}, the reaction is carried out under the conditions of 0.1 MPa-0.3 MPa and 25° C.-50° C.

10. The process for selectively reducing spinosyn J according to claim 6, wherein the hydrogenation materials in Step \textcircled{3}, the hydrogenation, tetrabutylammonium bromide, potassium hydroxide and water are added to a reactor, and the mixture is fully stirred to dissolve and then sealed in the reactor; nitrogen is introduced to the reactor to replace air in the reactor at least once; after adding ethyl bromide, the mixture inside the reactor is heated up to 40° C., the pressure rises to 0.3 Mpa by supplementing nitrogen, and the stirring speed is adjusted to 300 r/min; under these conditions, the mixture reacts for 5 h, is cooled down to room temperature, transferred to an enamel kettle, added with diethyl ether for crystallization, filtered, and dried at 60° C. to obtain spinetoram, a final product.
